(19) United States
(12) Patent Application Publication
(10) Pub. No.: US 2025/0000608 A1
(43) Pub. Date: Jan. 2, 2025

(54) TRACKING AND IMAGING COIL

(71) Applicant: Imricor Medical Systems Inc., Burnsville, MN (US)

(72) Inventors: Madison Winkelman, Lakeville, MN (US); Steven R. Wedan, Savage, MN (US)

(21) Appl. No.: 18/748,800

(22) Filed: Jun. 20, 2024

Related U.S. Application Data
(60) Provisional application No. 63/511,253, filed on Jun. 30, 2023.

(51) Int. Cl. A61B 90/00; A61B 34/20

(52) U.S. Cl. CPC A61B 90/39; A61B 34/20; A61B 2034/2051; A61B 2090/3954

(57) ABSTRACT

Implementing systems and methods for making a tracked device for use in a medical procedure. The methods comprise: obtaining a device configured to facilitate the medical procedure; arranging a first coil relative to a surface of the device so that (i) the first coil will partially surround or surround only a portion of the perimeter or circumference of the device and/or (ii) has a center axis that is not parallel to a center axis of the device; and disposing the first coil on or near the surface of the device such that the first coil is configured to receive a magnetic resonance signal from an imageable substance inside or outside the device during the medical procedure.

### CROSS-REFERENCE TO RELATED APPLICATIONS

The present application claims priory to and the benefit of U.S. Provisional Patent Application No. 63/511,253 which was filed on Jun. 30, 2023. The contents of this Provisional Patent Application is incorporated herein by reference in its entirety.

### BACKGROUND

Devices introduced into a patient utilizing magnetic resonance imaging (MRI) for image guidance often employ one or more magnetic resonance (MR) receive coil(s), or so-called “tracking coils” for the purpose of locating the device within the patient.

### SUMMARY

The present document concerns methods for making a tracked device for use in a medical procedure. The methods comprise: obtaining a device configured to facilitate the medical procedure; arranging a first coil relative to a surface of the device so that (i) the first coil will partially surround or surround only a portion of the perimeter or circumference of the device and/or (ii) has a center axis that is not parallel (e.g., perpendicular or otherwise angled relative) to a center axis of the device; and disposing the first coil on or near the surface of the device such that the first coil is configured to receive a magnetic resonance signal from an imageable substance inside or outside the device during the medical procedure.

The present document concerns trackable medical devices. The trackable medical devices comprise: a device configured to facilitate a medical procedure; and a first coil disposed on or near a surface of the device so that (i) the first coil partially surrounds or surrounds only a portion of the perimeter or circumference of the device, (ii) has a center axis that is not parallel or angled relative to a center axis of the device, and (iii) is configured to receive a magnetic resonance signal from an imageable substance inside or outside the device during a medical procedure.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 provides an illustration of a conventional tracked device.

FIGS. 2, 3, 7 and 11 each provides an illustration of a tracked device in accordance with the present solution.

FIGS. 4-6 each provides an illustration of a stack for a circuit board device in accordance with the present solution.

FIGS. 8-10 provide illustrations of different trace patterns for coils in accordance with the present solution.

FIG. 12 provides an illustration of another tracked device that can be deflected, bend or otherwise change shape.

FIG. 13 provides a flow diagram of a method for making a device in accordance with the present solution.

FIG. 14 provides an illustration of a magnetic resonance imaging system in which the devices of FIGS. 2-12 can be used.

FIG. 15 provides a flow diagram for performing a medical procedure using the present solution of FIG. 14.

FIG. 16 is a block diagram of a computing device.

FIG. 17 provides an illustration of another tracked device.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.”

In this document, when terms such as “first” and “second” are used to modify a noun, such use is simply intended to distinguish one item from another, and is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

When used in this document, terms such as “top” and “bottom,” “upper” and “lower”, “above” and “below”, “over” or “under”, or “front” and “behind,” are not intended to have absolute orientations but are instead intended to describe relative positions of various components with respect to each other. For example, a first component may be an “upper” component and a second component may be a “lower” component when a device of which the components are a part is oriented in a first direction. The relative orientations of the components may be reversed, or the components may be on the same plane, if the orientation of the structure that contains the components is changed. The claims are intended to include all orientations of a device containing such components.

Additional terms that are relevant to this disclosure will be defined at the end of this Detailed Description section.

Devices introduced into a patient utilizing MRI for image guidance often employ one or more MR receive coil(s), or so-called “tracking coils” for the purpose of locating the device within the patient. MR receive coils can be used in some scenarios as communication antenna. Tracking coils receive MR signals from nearby substances that react to the MR imaging sequence. A tracking coil's position can be estimated within the MRI system's FOV by scanning with specific MRI pulse sequences and receiving MR signals with the tracking coil. Subsequent signal processing can be used to estimate the x-, y-, and z-locations of the tracking coil.

Tracking coils can be difficult to manufacture consistently, as they commonly consist of either a helical coil of wire or a flex circuit closed upon itself to form a helix. In addition, the most MR sensitive volume (the volume from which the most MR signal can be received) is the center of a helical tracking coil. Device construction often limits this volume resulting in inadequate substances to receive a sufficient MR signal. Consequently, the desired receive volume often must include regions outside the coil. Furthermore, the miniature nature of helix and helical tracking coils results in limited FOVs (the volume from which any appreciable MR signal can be received), limiting their usefulness for imaging purposes. The terms helix and helical are used interchangeably herein.

The present invention is directed towards devices used in medical procedures such as cardiac ablation catheters, diagnostic catheters, endomyocardial ablation catheters, biopsy catheters, delivery systems, or the like. The present solution will be described below in relation to catheters. However, the present solution is not limited in this regard. The particulars of the present solution will become evident as the discussion progresses.

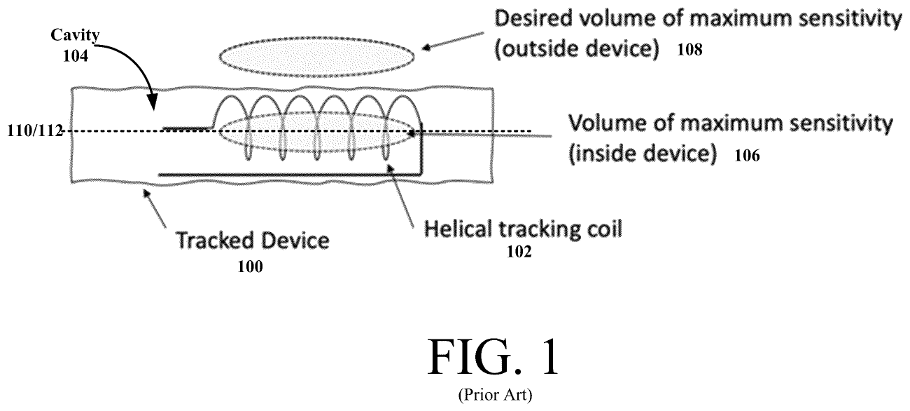

Referring now to FIG. 1, there is provided an illustration of a conventional tracked device 100. Device 100 can include, but is not limited to, a catheter usable in a magnetic resonance imaging system such as that disclosed in U.S. Pat. No. 11,318,280 to Weiss et al. (“the '280 Patent”). Device 100 may be configured to apply energy to an object and the magnetic resonance imaging system to localize the device 100. A helical tracking coil 102 is disposed inside a space, cavity or chamber 104 of the device 100. The tracking coil 102 surrounds a volume of maximum sensitivity 106 inside the device 100. The volume 106 may be provided by a lumen of a catheter. In this case, the tracking coil 102 is wrapped or otherwise disposed around the lumen. A fluid (e.g., saline) flows inside the lumen from which the tracking coil can receive an MRI signal. The received MRI signal can be processed to estimate the x-, y-, and z-locations of the tracking coil 102.

Conventional device 100 suffers from certain drawbacks. For example, helix or helical tracking coils are difficult to consistently manufacture. Additionally, the most MR sensitive volume (i.e., the volume from which the most MR signal can be received) is the center of a helical tracking coil 102. The construction of device 100 limits the volume and substances resulting in inadequate MR signal(s) in the sensitive volume of the tracking coil 102. Consequently, the desired receive volume 108 often must include region(s) outside the tracking coil 102. Furthermore, the miniature nature of tracking coil 102 results in a limited FOV (i.e., the volume from which any appreciable MR signal can be received), limiting its usefulness for imaging purposes.

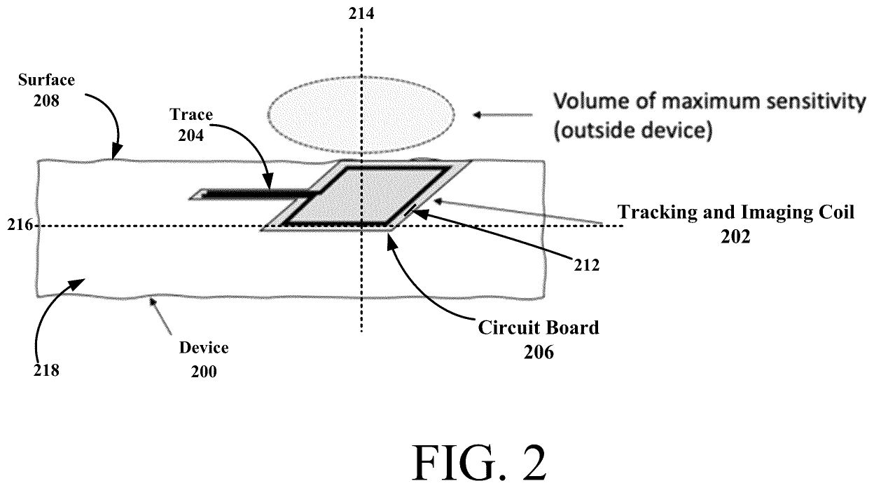

Referring now to FIG. 2, there is provided an illustration of a device 200 in accordance with the present solution. Device 200 can include, but is not limited to, a catheter usable in a magnetic resonance imaging system. In the catheter scenario, device 200 is generally cylinder in shape. The present solution is not limited in this regard. Device 200 can be other shapes in accordance with any given application (e.g., planar, rectangular, etc.).

Device 200 may be configured to apply energy to an object and the magnetic resonance imaging system to localize the device 200. In this regard, a tracking and imaging coil 202 is disposed on, coupled to, or located adjacent or proximate to an outside surface 208 of the device 200. An insulative material may be disposed on the tracking and imaging coil 202. The insulative material can include, but is not limited to, epoxy, glue, adhesive, a heat shrink material and/or other material that is suitable for creating a barrier between an external environment and the coil. The coupling of coil 202 to surface 208 can be achieved using, for example, an adhesive and/or other coupling means.

Coil 202 comprises a non-helical coil formed by a trace 204 printed or disposed on a layer of the circuit board 206. The trace 204 can comprise a conductive substance such as ink, film and/or foil. Any known or to be known technique for making a trace on a substrate can be used herein without limitation. Coil is configured to receive MR signals. The circuit board 206 may comprise a rigid or flexible material or substrate. This coil design allows for more manufacturable solutions as the circuit of the coil 202 does not need to close upon itself and can facilitate improved device tracking and imaging around the device 200.

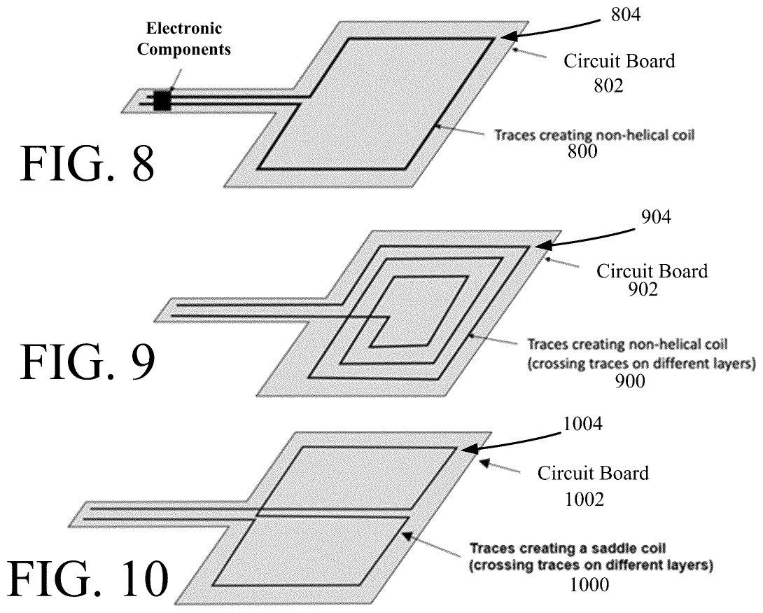

One or more other electronic components 212 may also optionally be disposed on the circuit board 206 in accordance with any given application. The electronic components 212 can include, but are not limited to, passive components (e.g., resisters, capacitors, inductors, etc.), a tuning circuit, an impedance matching circuit, sensor(s), processor(s), piezoelectric components, and/or wireless communication device(s). The present solution is not limited to the location of the electronic components 212 shown in FIG. 2. The electronic components could, for example, be positioned as shown in FIG. 8

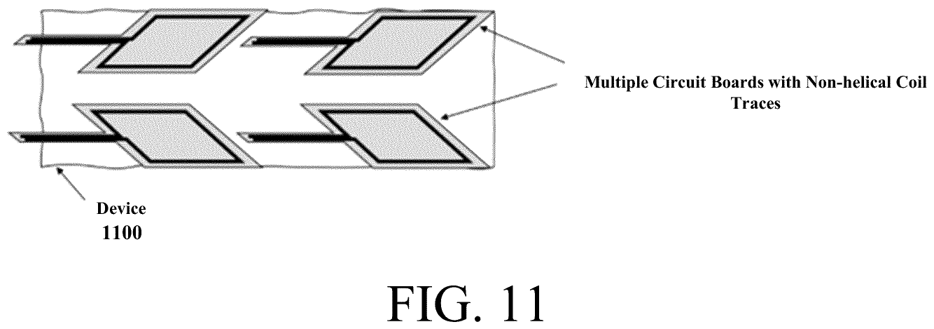

Although a single coil 202 and/or circuit board 206 is shown in FIG. 2, the present solution is not limited in this regard. Any number of coils 202 can be provided with the circuit board 206 in accordance with any given application. Similarly, any number of circuit boards can be provided in accordance with any given application. The circuit boards may be disposed on the device, for example as shown in FIG. 11, to provide an array for imaging purposes. For example, the array is provided to sense imaging.

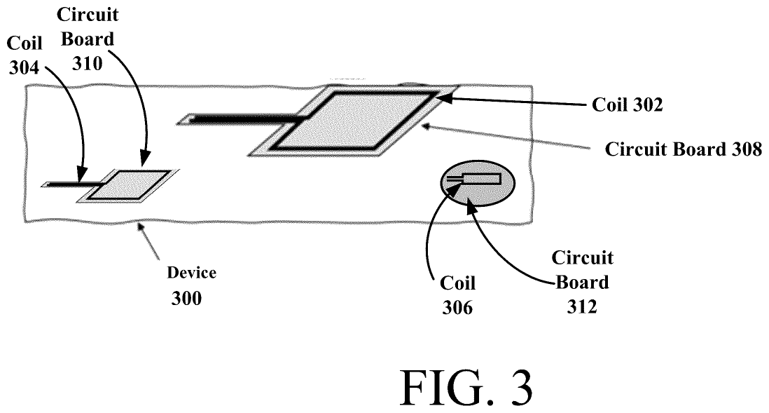

An illustration showing a device 300 with three coils 302, 304, 306 disposed therein is provided in FIG. 3. The coils 302, 304, 306 are each disposed on a respective substrate of the circuit board 308, 310, 312. The present solution is not limited to this coil-circuit board arrangement. For example, in some scenarios, two or more of the coils may reside on the same substrate and/or circuit board. The coils 302, 304, 306 are of different sizes and/or shapes. The circuit boards 308, 310, 312 are also of different sizes and/or shapes. The total number, shapes and/or sizes of the coils and/or circuit boards can be selected in accordance with a particular application. For example, the total number, shapes and/or sizes of the coils and/or circuit boards can be selected to focus a FOV, expand the FOV, increase a receive signal sensitivity, and/or reduce the receive signal sensitivity.

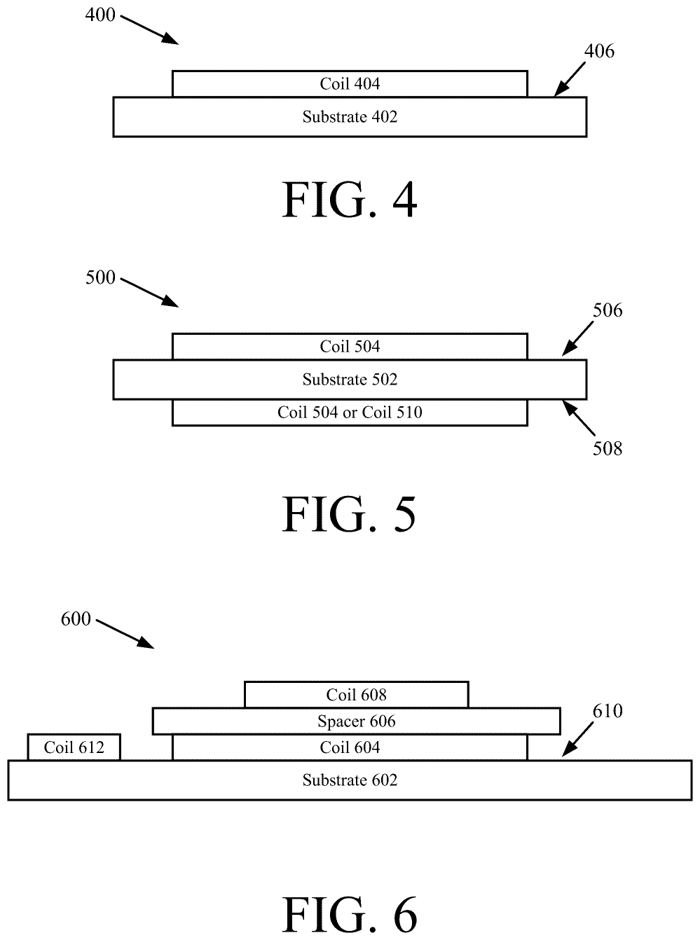

Two or more coils can be located in and/or on the same or differing layers of the circuit board. Illustrations of stacks for circuit boards 400, 500, 600 are provided in FIGS. 4-6. Any of the coil and substrate configurations of FIGS. 4-6 can be used in addition to or in the alternative to circuit boards 206, 308, 310, 312 of FIGS. 2-3. Circuit board 400 comprises a substrate 402 with a first surface 406 on which a coil 404 is a disposed. Circuit board 500 comprises a substrate 502 with a first surface 506 on which a first coil 504 is at least partially disposed and a second surface 508 on which the first coil 504 is at least partially disposed or a second coil 510 is a disposed. Circuit board 600 comprises a substrate 602 with a surface 610 on which a first coil 604 and a second coil 612 is disposed. A third coil 608 is disposed above the first coil 604. The first and third coils 604, 608 are spaced apart by a spacer 606. The spacer 606 may be the same as, similar to or different than substrate 602. The spacer 606 may be formed of a plastic material or other material that electrically isolates the coils from each other and/or allows an MR signal to be received by coils 604, 608, for example, via an imageable substance located inside and/or outside the device 600. The imageable substance can include, but is not limited to, a fluid, a gel, a plastic or other substances materials that produces an MRI signal. As noted above, other electronic components can be provided on the circuit. These other electronic components are not shown in FIGS. 4-6 simply for ease of illustration.

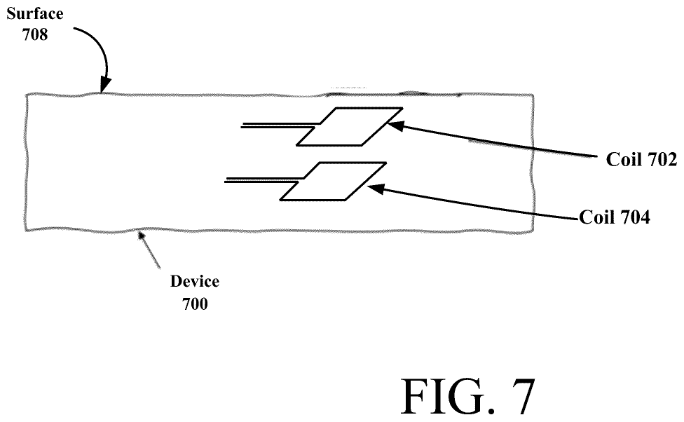

The present solution is not limited to the circuit board based coil designs of FIGS. 2-6. Coil(s) 702, 704 could alternatively be disposed directly on or near the surface 708 of the device, as shown in FIG. 7. Surface 708 may be an inner surface or an outer surface of the device. In this case, the surface may be at least partially formed of a plastic or other non-conductive material. Each coil 702, 704 can include a trace or a wire. The trace can be deposited or printed on or within the device 700. The wire may be coupled to the device 700 via an adhesive or other coupling means.

The coils of the device(s) can also have the same or different trace patterns. Illustrative trace patterns 800, 900, 1000 for coils are shown in FIGS. 8-10. The potential patterns are numerous and not limited to those shown. Each of the trace coils comprises a non-helical coil or a saddle coil.

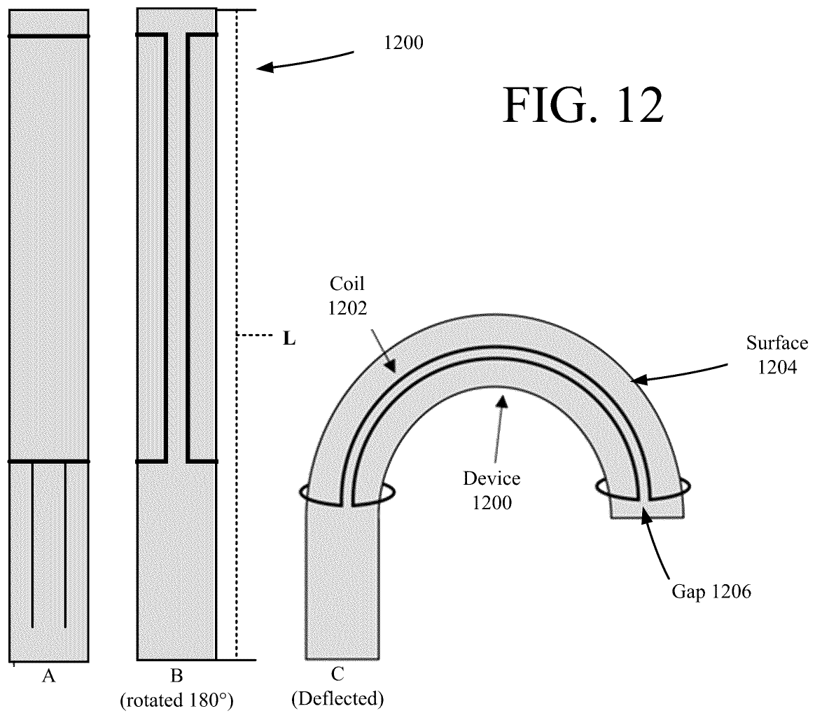

An illustration of yet another device 1200 is provided in FIG. 12. Device 1200 is configured to bend, deform, deflect or otherwise change shape. A coil 1202 is disposed on or near surface 1204 of the device 1200. Coil 1202 comprises a non-helical coil including, but not limited to, the designs of FIGS. 2-11 that extends partially around the circumference of the device 1200 which has a cylindrical shape and also extends along the elongated length L of the device 1200. A gap 1206 is provided between the left and right sides of the coil 1202. Electrical components may be provided on the circuit but are not shown.

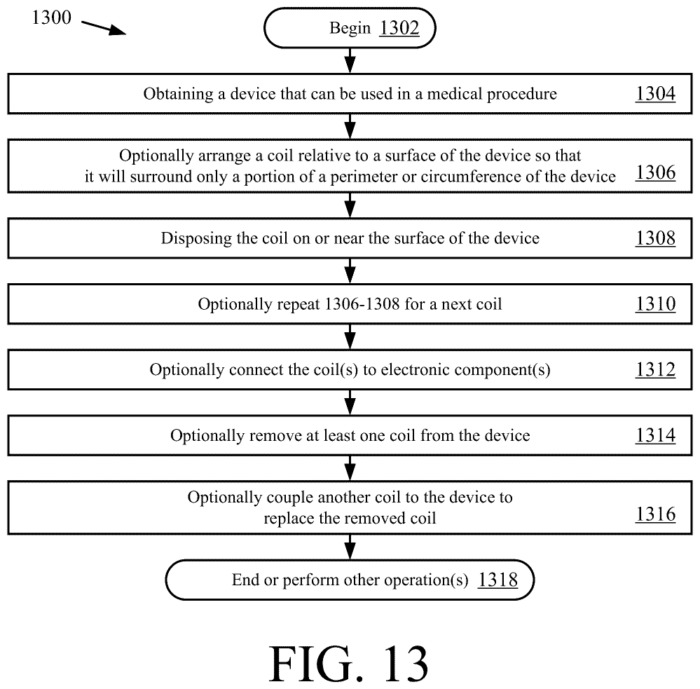

FIG. 13 provides a flow diagram of an illustrative method 1300 of making a device in accordance with the present solution. Method 1300 begins with 1302 and continues with 1304 where a device is obtained that can be used in a medical procedure. The device can include, but is not limited to, cardiac ablation catheters, diagnostic catheters, endomyocardial ablation catheters, biopsy catheters, and/or delivery systems.

In 1306, a coil is optionally arranged relative to a surface of the device so that (i) the coil will partially surround or surround only a portion of the perimeter or circumference of the device and/or (ii) has a center axis (e.g., axis 214 of FIG. 2) that is not parallel (e.g., angled relative or perpendicular) to a center axis (e.g., axis 216 of FIG. 2) the device, and/or (iv) has a center axis that is not parallel (e.g., angled or perpendicular) to a center axis of a channel in which the magnetic resonance substance resides or will reside. The magnetic resonance substance constitutes an imageable substance. In conventional solutions, the central axis of the helical coil (e.g., axis 110 of FIG. 1) is parallel with the central axis of the channel in which the magnetic resonance substance resides (e.g., axis 112 of FIG. 1). This is an important distinction because the present solution provides a simpler and less costly design while still allowing for the device tracking using coil(s) that receive magnetic resonance signals from the substance.

The coil may comprise a wire (e.g., wire 702 or 704 of FIG. 7) or a trace (e.g., trace 204 of FIG. 2, 302 of FIG. 3, 304 of FIG. 3, 306 of FIG. 3, 404 of FIG. 4, 504 of FIG. 5, 510 of FIG. 5, 604 of FIG. 6, 608 of FIG. 6, 612 of FIG. 6, 800 of FIG. 8, 900 of FIG. 9, or 1000 of FIG. 10,) coupled to a substrate (e.g., substrate 402 of FIG. 4, 502 of FIG. 5, 602 of FIG. 6, 708 of FIG. 6) or circuit board (e.g., circuit board 206 of FIG. 2, 308 of FIG. 3, 310 of FIG. 3, 312 of FIG. 3, 802 of FIG. 8, 902 of FIG. 9, or 1002 of FIG. 10). The coil/substrate structures and the coil/circuit board structures are referred to herein as coil structure.

In 1308, the coil is coupled on or near to the surface (e.g., surface 208 of FIG. 2, 708 of FIG. 7 or surface 1204 of FIG. 12) of the device. In some scenarios, the coupling can be achieved using a trace disposition or printing process such that the coil is disposed or printed directly on a surface on or near the outside of the device. In other scenarios, the coupling is achieved using an adhesive, tape or other coupling means. The adhesive, tape or other coupling means may be disposed on the wire, the surface of the device, and/or a bottom surface of a coil structure. The adhesive, tape or coupling means can be selected to allow the wire coil or coil structure to be removable from the device so that it can be interchanged with another wire coil or coil structure at any time. In some scenarios, the coil structure may be in the form of a pad.

The operations of blocks 1306-1308 can be repeated for a next coil as shown by 1310. Any number of coils can be coupled to the device. The coils can be of the same type or of different types. For example, the coils can comprise wire coils, coil structure, or both wire and coil structures. The coils could also have the same or different shapes and/or sizes. The coils could be arranged in a given pattern on the surface of the device to provide, for example, an array for imaging purposes (e.g., sense imaging).

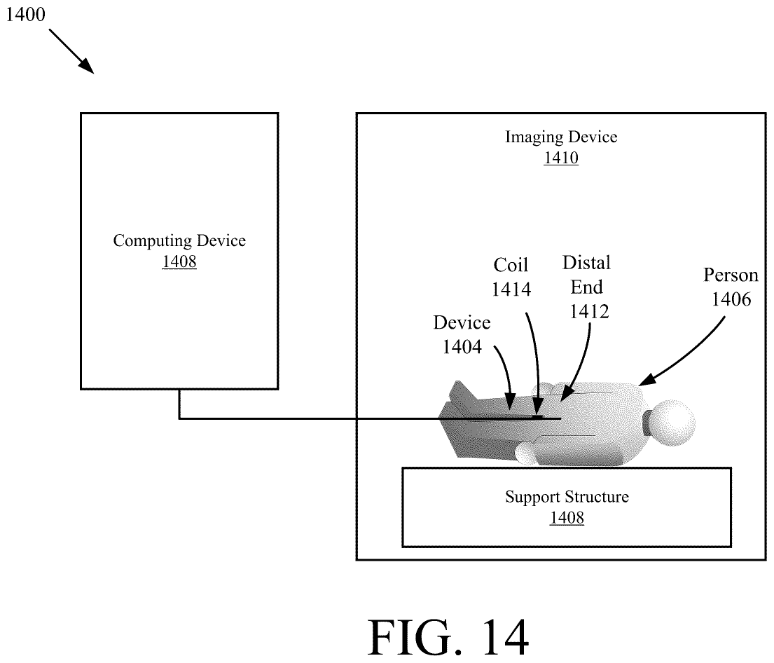

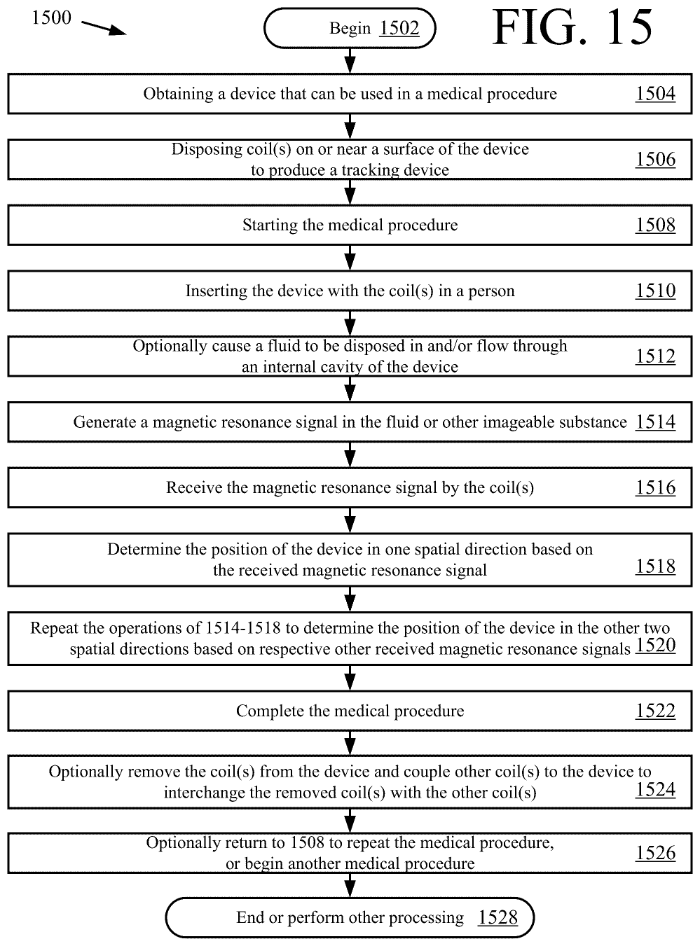

The result of the coupling operation(s) produces a tracked device having one or more coils along a length of the device. For example, the coil(s) may be located at its distal end (e.g., distal end 1412 of FIG. 14), middle portion, and/or proximal end. The tracked device can include, but is not limited to, device 200 of FIG. 2, device 300 of FIG. 3, device 700 of FIG. 7, device 1100 of FIG. 11, device 1200 of FIG. 12, or device 1404 of FIG. 14.

In block 1312, the coil(s) may optionally be connected to electronic component(s). For example, each coil may be solder connected to a tuning and matching network.

In block 1314, at least one coil is optionally removed from the tracked device. Another coil can be optionally coupled to the device for replacing the removed coil, as shown by 1316. The replacement coil can be the same as the removed coil or have at least one characteristic that is different than the removed coil. The characteristic(s) can include, but are not limited to, shape, size, and/or coil type (e.g., non-helical, saddle coil, wire coil, trace coil, single layer trace coil, or multi-layer trace coil). Subsequently, 1318 is performed where method 1300 ends or other operations are performed.

FIG. 14 provides an illustration of a magnetic resonance imaging system 1400 in which the present solution can be used. System 1400 is generally configured to localize a device 1404 which has been inserted into a person 1406 located on a support structure 1408 within an imaging device 1310. Device 1404 can be the same as or similar to device 200 of FIG. 2, 300 of FIG. 3, 400 of FIG. 4, 500 of FIG. 5, 600 of FIG. 6, 700 of FIG. 7, 800 of FIG. 8, 900 of FIG. 9, 1000 of FIG. 10, 1100 of FIG. 11, or 1200 of FIG. 12. Device 1304 has a coil 1314 disposed on its distal end 1312. Device 1404 is electrically and communicatively coupled to computing device 1408.

During operation, the imaging device 1410 performs operations to generate a magnetic resonance signal in a substance present in or outside of the device 1404. The magnetic resonance signal is detected by the coil 1414 and passed to the computing device 1408. The computing device 1408: receives the magnetic resonance signal from the coil 1414; and processes the received magnetic resonance signal to determine the position of the device 1404 within the person 1406.

Referring now to FIG. 15, there is provided a flow diagram of an illustrative method 1500 for using the device of the present solution. Method 1500 begins with 1502 and continues with 1504 where a device is obtained that can be used in accordance with a given application, such as in a medical procedure. In the medical procedure scenario, the device may comprise a catheter.

One or more coils are disposed on or near a surface of the device in block 1506. The result of this coupling produces a tracking device. The tracking device can include, but is not limited to, a catheter, a drug delivery system, device 200 of FIG. 2, device 300 of FIG. 3, device 400 of FIG. 4, device 500 of FIG. 5, device 600 of FIG. 6, device 700 of FIG. 7, device 800 of FIG. 8, device 900 of FIG. 9, device 1000 of FIG. 10, device 1100 of FIG. 11, device 1200 of FIG. 12, and/or device 1404 of FIG. 14. The coil(s) can be coupled, deposited or printed directly on a surface (e.g., surface 208 of FIG. 2, 708 of FIG. 7 or 1204 of FIG. 12) of the device or a surface of a structure near the device. The coil(s) can include, but are not limited to, a non-helical coil and/or a saddle coil. For example, a first coil comprises (i) a wire coil coupled directly to the surface of the device, (ii) a trace coil coupled directly to the surface of the device, or (iii) a trace coil disposed on a flexible circuit board. The present solution is not limited to the particulars of this example.

Additionally or alternatively, the coil(s) may be combined with a printed circuit board to provide a tracking coil structure that can be positioned proximate to the device and/or adhered or otherwise affixed to the device. The coil may be electrically connected to an electronic component (e.g., electronic component 212 of FIG. 2) of the device. The electronic component can include, but is not limited to, a tuning and matching circuit. The electronic component may be coupled to or disposed on the printed circuit board along with the coil(s).

In all cases, the coil(s) partially surround(s) or surround(s) only a portion of the circumference of the device (e.g., if the device has a circular cross-sectional profile) or perimeter of the device (e.g., if the device has a straight-sided cross-sectional profile). Consequently, the central axis of the coil(s) (e.g., axis 214 of FIG. 2) is not parallel (e.g., perpendicular) to the central axis of the device. When the imageable substance comprises a fluid flowing in a channel inside the device, the central axis of the coil(s) are not parallel (e.g., perpendicular) to the central axis of the channel through which the fluid resides and/or flows (e.g., axis 216 of FIG. 2). In conventional solutions, the central axis of the helical coil (e.g., axis 110 of FIG. 1) is parallel with the central axis of the channel in which the magnetic resonance substance resides (e.g., axis 112 of FIG. 1). This is an important distinction because the present solution provides a simpler and less costly design while still allowing for the device tracking using coil(s) that receive magnetic resonance signals from the substance.

In some scenarios, first and second coils are coupled on or near the surface of the device. The second coil may be the same as or different than the first coil. The first coil and the second coil may be arranged on the surface of the device to provide an array for an imaging device during a medical procedure. The present solution is not limited to the particulars of this example.

In 1508, a medical procedure is started. During the medical procedure, operations of blocks 1510-1520 are performed. These operations may involve: inserting the device into a person (and optionally steering the distal end of the device to a desired location in the person's body); optionally causing a fluid to be disposed in and/or flow through an internal space, cavity or chamber (e.g., space, cavity or chamber 218 of FIG. 2) of the device; generate a magnetic resonance signal in the fluid and/or other imageable substance; receive the magnetic resonance signal by the coil(s); and determine the position of the device in one spatial direction (e.g., the x-spatial direction) based on the received magnetic resonance signal. The operations of blocks 1514-1518 are repeated to determine the position of the device in the other two spatial directions (e.g., the y- and z-spatial directions). The medical procedure is completed in 1522.

Method 1500 may continue with blocks 1524 and/or 1526 where optional operations may be performed. The optional operations involve: removing the coil(s) from the device and couple other coil(s) to the device to interchange the removed coil(s) with other coil(s); and/or returning to block 1508 to repeat the medical procedure or begin another medical procedure. Subsequently, 1528 is performed where method 1500 ends or other operations are performed.

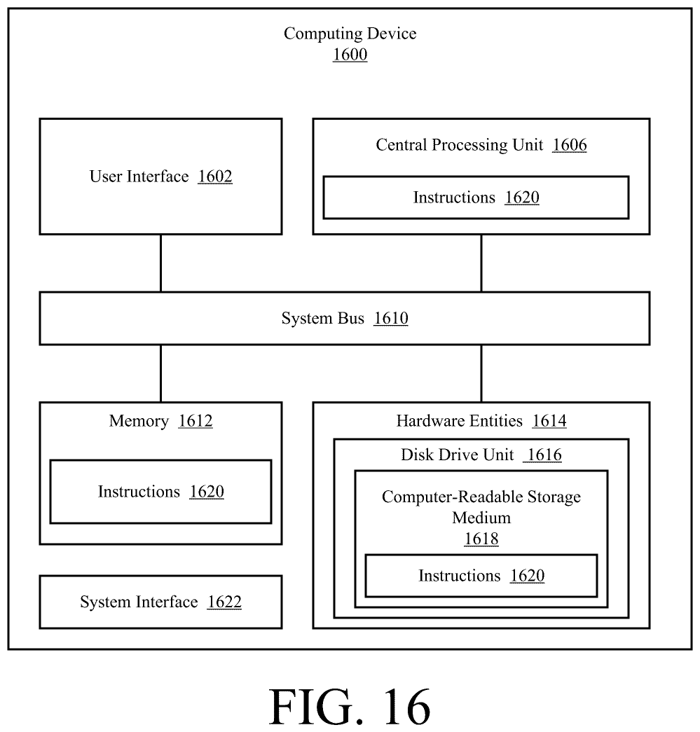

Referring now to FIG. 16, there is provided a block diagram of an exemplary embodiment of the computing device 1600. Computing device 1408 of FIG. 14 can be the same as or similar to computing device 1600. As such, the discussion of computing device 1600 is sufficient for understanding computing device 1408.

Computing device 1600 can include, but is not limited to, a notebook, a desktop computer, a laptop computer, a personal digital assistant, and a tablet PC. Some or all the components of the computing device 1600 can be implemented as hardware, software and/or a combination of hardware and software. The hardware includes, but is not limited to, one or more electronic circuits.

Computing device 1600 may include more or less components than those shown in FIG. 16. However, the components shown are sufficient to disclose an illustrative embodiment implementing the present invention. The hardware architecture of FIG. 16 represents one embodiment of a representative computing device configured to facilitate spatial feature data analysis in an efficient manner. As such, the computing device 1600 of FIG. 16 implements improved methods for spatial feature data analysis in accordance with embodiments of the present invention.

As shown in FIG. 16, computing device 1600 includes a system interface 1622, a user interface 1602, a Central Processing Unit (CPU) 1606, a system bus 1610, a memory 1612 connected to and accessible by other portions of computing device 1600 through system bus 1610, and hardware entities 1614 connected to system bus 1610. At least some of the hardware entities 1614 perform actions involving access to and use of memory 1612, which can be a Random Access Memory (RAM), a disk driver and/or a Compact Disc Read Only Memory (CD-ROM).

System interface 1622 allows the computing device 1600 to communicate directly or indirectly with external communication devices (e.g., imaging device 1410 of FIG. 14). If the computing device 1600 is communicating indirectly with the external communication device, then the computing device 1600 is sending and receiving communications through a common network (e.g., the Internet or Intranet).

Hardware entities 1614 can include a disk drive unit 1616 comprising a computer-readable storage medium 1618 on which is stored one or more sets of instructions 1620 (e.g., software code) configured to implement one or more of the methodologies, procedures, or functions described herein. The instructions 1620 can also reside, completely or at least partially, within the memory 1612 and/or within the CPU 1606 during execution thereof by the computing device 1600. The memory 1612 and the CPU 1606 also can constitute machine-readable media. The term “machine-readable media”, as used here, refers to a single medium or multiple media (e.g., a centralized or distributed database, and/or associated caches and servers) that store the one or more sets of instructions 1620. The term “machine-readable media”, as used here, also refers to any medium that is capable of storing, encoding or carrying a set of instructions 1620 for execution by the computing device 1600 and that cause the computing device 1600 to perform any one or more of the methodologies of the present disclosure.

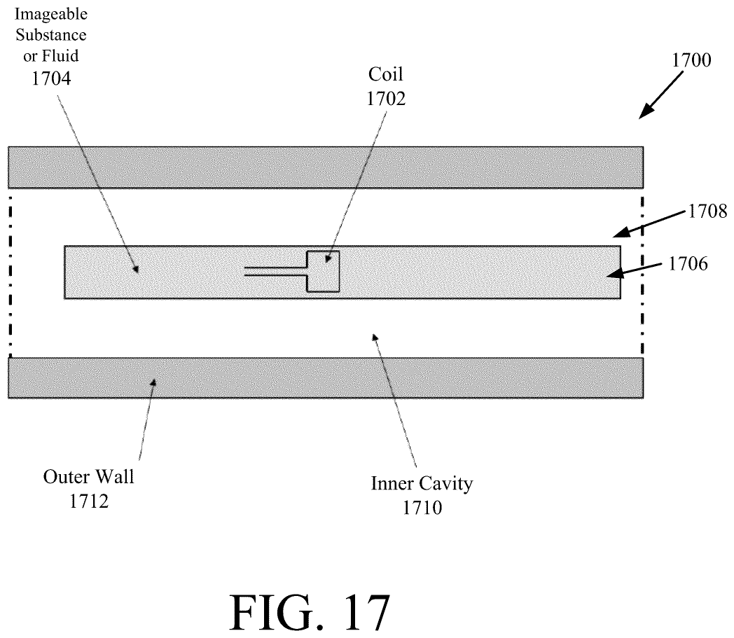

It should be noted that the phrase “on or near the surface” covers the scenario shown in FIG. 17. In FIG. 17, the coil 1702 is coupled to or disposed directly on a surface 1706 surrounding imageable substance or fluid 1702. The surface 1706 may be, for example, a surface of tubing 1708 filled with the imageable substance or fluid 1702. Tubing 1708 may be located with an inner cavity 1710 of the device 1700 defined by outer wall 1712. Tubing 1708 may or may not be aligned with a center axis of the device 1700 and/or a center axis of the inner cavity 1710. The inner cavity 1710 can be an empty space, filled with other components of device 1700, or filled with solid material(s).

Terminology that is relevant to this disclosure includes:

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices. A computer program product is a memory device with programming instructions stored on it.

The features and functions described above, as well as alternatives, may be combined into many other different systems or applications. Various alternatives, modifications, variations or improvements may be made by those skilled in the art, each of which is also intended to be encompassed by the disclosed embodiments.

Without excluding further possible embodiments, certain example embodiments are summarized in the following clauses:

Clause 1. A method for making a tracked device for use in a medical procedure, comprising: obtaining a device configured to facilitate the medical procedure; arranging a first coil relative to a surface of the device so that (i) the first coil will partially surround or surround only a portion of the perimeter or circumference of the device and/or (ii) has a center axis that is not parallel (e.g., perpendicular or otherwise angled) to a center axis of the device; and disposing the first coil on or near the surface of the device such that the first coil is configured to receive a magnetic resonance signal from an imageable substance inside or outside the device during the medical procedure.

Clause 2. The method according to clause 1, wherein the device comprises an internal space for receiving a fluid and the first coil is configured to receive the magnetic resonance signal from the fluid.

Clause 3. The method according to any of the preceding clauses, wherein the device comprises a catheter or a drug delivery system.

Clause 4. The method according to any of the preceding clauses, wherein the first coil comprises a non-helical coil or a saddle coil.

Clause 5. The method according to any of the preceding clauses, wherein the disposing comprises attaching a wire coil directly to the surface of the device.

Clause 6. The method according to any of the preceding clauses, wherein the disposing comprises disposing a trace coil directly on the surface of the device.

Clause 7. The method according to any of the preceding clauses, wherein the first coil comprises a non-helical coil extending an elongated length of the device configured to bend, deform, deflect or otherwise change shape.

Clause 8. The method according to any of the preceding clauses, wherein the disposing comprises affixing a coil structure to the surface of the device, the coil structure comprising a trace coil disposed on a flexible circuit board.

Clause 9. The method according to any of the preceding clauses, further comprising electrically connecting the first coil to an electronic component of the device.

Clause 10. The method according to any of the preceding clauses, wherein the electronic component comprises a tuning and matching circuit.

Clause 11. The method according to any of the preceding clauses, further comprising coupling at least one second coil on or near the surface of the device.

Clause 12. The method according to any of the preceding clauses, wherein the at least one second coil is the same as or different than the first coil.

Clause 13. The method according to any of the preceding clauses, wherein the first coil and the at least one second coil are arranged on the surface of the device to provide an array for an imaging device during the medical procedure.

Clause 14. The method according to any of the preceding clauses, further comprising removing the first coil from the device and coupling a second coil to the device to interchange the removed first coil with the second coil.

Clause 15. A trackable medical device, comprising: a device configured to facilitate a medical procedure; and a first coil disposed on or near a surface of the device so that (i) the first coil partially surrounds or surrounds only a portion of the perimeter or circumference of the device, (ii) has a center axis that is not parallel to a center axis of the device, and (iii) is configured to receive a magnetic resonance signal from an imageable substance inside or outside the device during a medical procedure.

Clause 16. The trackable medical device according to clause 15, wherein the device comprises an internal space for receiving a fluid and the first coil is configured to receive the magnetic resonance signal from the fluid.

Clause 17. The trackable medical device according to any preceding clause, wherein the device comprises a catheter or a drug delivery system.

Clause 18. The trackable medical device according to any preceding clause, wherein the first coil comprises a non-helical coil or a saddle coil.

Clause 19. The trackable medical device according to any preceding clause, wherein the first coil comprises a wire coil coupled directly to the surface of the device.

Clause 20. The trackable medical device according to any preceding clause, wherein the first coil comprises a trace coil disposed directly on the surface of the device.

Clause 21. The trackable medical device according to any preceding clause, wherein the first coil comprises a trace coil disposed on a flexible circuit board.

Clause 22. The trackable medical device according to any preceding clause, further comprising a tuning and matching circuit electrically connected to the first coil.

Clause 23. The trackable medical device according to any preceding clause, further comprising at least one second coil disposed on or near the surface of the device.

Clause 24. The trackable medical device according to any preceding clause, wherein the at least one second coil is the same as or different than the first coil.

Clause 25. The trackable medical device according to any preceding clause, wherein the first coil and the at least one second coil are arranged on the surface of the device to provide an array for an imaging device during the medical procedure.

Clause 26. The trackable medical device according to any preceding clause, wherein the first coil is interchangeable with at least one other second coil.

Clause 27. The trackable medical device according to any preceding clause, wherein the first coil comprises a non-helical coil extending an elongated length of the device configured to bend, deform, deflect or otherwise change shape.

The breadth and scope of this disclosure should not be limited by any of the above-described example embodiments, but should be defined only in accordance with the following claims and their equivalents.

We claim:

1. A method for making a tracked device for use in a medical procedure, comprising:
obtaining a device configured to facilitate the medical procedure;
arranging a first coil relative to a surface of the device so that (i) the first coil will partially surround or surround only a portion of the perimeter or circumference of the device and/or (ii) has a center axis that is not parallel to a center axis of the device; and
disposing the first coil on or near the surface of the device such that the first coil is configured to receive a magnetic resonance signal from an imageable substance inside or outside the device during the medical procedure.

2. The method according to claim 1, wherein the device comprises an internal space for receiving a fluid and the first coil is configured to receive the magnetic resonance signal from the fluid.

3. The method according to claim 1, wherein the device comprises a catheter or a drug delivery system.

4. The method according to claim 1, wherein the first coil comprises a non-helical coil or a saddle coil.

5. The method according to claim 1, wherein the disposing comprises attaching a wire coil directly to the surface of the device.

6. The method according to claim 1, wherein the disposing comprises disposing a trace coil directly on the surface of the device.

7. The method according to claim 1, wherein the first coil comprises a non-helical coil extending an elongated length of the device configured to bend, deform, deflect or otherwise change shape.

8. The method according to claim 1, wherein the disposing comprises affixing a coil structure to the surface of the device, the coil structure comprising a trace coil disposed on a flexible circuit board.

9. The method according to claim 1, further comprising electrically connecting the first coil to an electronic component of the device.

10. The method according to claim 9, wherein the electronic component comprises a tuning and matching circuit.

11. The method according to claim 1, further comprising coupling at least one second coil on or near the surface of the device.

12. The method according to claim 11, wherein the at least one second coil is the same as or different than the first coil.

13. The method according to claim 11, wherein the first coil and the at least one second coil are arranged on the surface of the device to provide an array for an imaging device during the medical procedure.

14. The method according to claim 1, further comprising removing the first coil from the device and coupling a second coil to the device to interchange the removed first coil with the second coil.

15. A trackable medical device, comprising:
a device configured to facilitate a medical procedure; and
a first coil disposed on or near a surface of the device so that (i) the first coil partially surrounds or surrounds only a portion of the perimeter or circumference of the device, (ii) has a center axis that is not parallel to a center axis of the device, and (iii) is configured to receive a magnetic resonance signal from an imageable substance inside or outside the device during a medical procedure.

16. The trackable medical device according to claim 15, wherein the device comprises an internal space for receiving a fluid and the first coil is configured to receive the magnetic resonance signal from the fluid.

17. The trackable medical device according to claim 15, wherein the device comprises a catheter or a drug delivery system.

18. The trackable medical device according to claim 15, wherein the first coil comprises a non-helical coil or a saddle coil.

19. The trackable medical device according to claim 15, wherein the first coil comprises a wire coil coupled directly to the surface of the device.

20. The trackable medical device according to claim 15, wherein the first coil comprises a trace coil disposed directly on the surface of the device.

21. The trackable medical device according to claim 15, wherein the first coil comprises a trace coil disposed on a flexible circuit board.

22. The trackable medical device according to claim 15, further comprising a tuning and matching circuit electrically connected to the first coil.

23. The trackable medical device according to claim 15, further comprising at least one second coil disposed on or near the surface of the device.

24. The trackable medical device according to claim 23, wherein the at least one second coil is the same as or different than the first coil.

25. The trackable medical device according to claim 23, wherein the first coil and the at least one second coil are arranged on the surface of the device to provide an array for an imaging device during the medical procedure.

26. The trackable medical device according to claim 15, wherein the first coil is interchangeable with at least one other second coil.

27. The trackable medical device according to claim 13, wherein the first coil comprises a non-helical coil extending an elongated length of the device configured to bend, deform, deflect or otherwise change shape.
